(19) United States
(12) Patent Application Publication
(10) Pub. No.: US 2022/0211574 A1
(43) Pub. Date: Jul. 7, 2022

(54) SMART EXERCISE MAT

(71) Applicant: Therabody, Inc., Los Angeles, CA (US)

(72) Inventors: Eduardo Merino, Los Angeles, CA (US); Benjamin Nazarian, Los Angeles, CA (US); Jaime Sanchez Solana, Los Angeles, CA (US)

(21) Appl. No.: 17/704,505

(22) Filed: Mar. 25, 2022

Related U.S. Application Data
(63) Continuation-in-part of application No. 17/066,230, filed on Oct. 8, 2020.
Application No. 17/066,230 is a continuation-in-part of application No. 16/796,143, filed on Feb. 20, 2020, now Pat. No. 10,940,081.
(60) Provisional application No. 63/165,858, filed on Mar. 25, 2021.
(60) Provisional application No. 62/912,392, filed on Oct. 8, 2019.
(60) Provisional application No. 62/899,098, filed on Sep. 11, 2019.
(60) Provisional application No. 62/844,424, filed on May 7, 2019.

(51) Int. Cl. A61H 23/02; A61H 7/00

(52) U.S. Cl. CPC A61H 23/0254; A61H 2205/081; A61H 2201/0207; A61H 7/001

(57) ABSTRACT

A mat assembly that includes a mat that includes a top layer and a bottom layer, a plurality of vibration motors positioned between the top layer and the bottom layer, a plurality of sensors associated with the mat, a removable battery pack, and a controller. In use, a first set of vibration motors within the plurality of vibration motors is configured to be actuated when a first one of the plurality of sensors is actuated.

### CROSS-REFERENCE TO RELATED APPLICATIONS

This application claims the benefit of U.S. Provisional Application No. 63/165,858, filed Mar. 25, 2021. This application is also a continuation-in-part of U.S. application Ser. No. 17/066,230, filed on Oct. 8, 2020, which is a continuation-in-part of U.S. patent application Ser. No. 16/796,143, filed Feb. 20, 2020, now U.S. Pat. No. 10,940,081, which claims the benefit of U.S. Provisional Application No. 62/912,392, filed Oct. 8, 2019, U.S. Provisional Application No. 62/899,098, filed Sep. 11, 2019 and U.S. Provisional Application No. 62/844,424, filed May 7, 2019, all of which are incorporated by reference herein in their entireties.

### FIELD OF THE INVENTION

The present invention relates to an exercise mat, and more particularly to a smart exercise mat.

### BACKGROUND OF THE INVENTION

Yoga mats or other mats for exercise, relaxation, stretching and the like are known. The present invention provides advantages and improvements over prior art exercise mats.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with a first aspect of the present invention there is provided a mat assembly that includes a mat having a top layer and a bottom layer, a plurality of vibration motors positioned between the top layer and the bottom layer, a plurality of sensors associated with the mat, a removable battery pack, a controller, and a graphical user interface positioned on the top layer. In use, a first set of vibration motors (a set can be one vibration motor) within the plurality of vibration motors is actuated when a first one of the plurality of sensors is actuated. The sensor is actuated when it senses what it is programmed or otherwise meant to sense or detect, such as the weight of a user. For example, the sensor may be a strain gauge that is “actuated” when a user places weight on the strain gage or on the mat at a position above the strain gauge. Other weight sensors include capacitance, hydraulic and pneumatic. However, any type of sensor that has the ability to sense the weight or presence of the user thereabove or close thereto is within the scope of the present invention.

The present invention is a smart mat assembly that can be used independently or as part of a wellness ecosystem that can or may include percussive therapy devices, see, e.g., U.S. Patent Publication No. 2020/0261307 (the “'307 publication”), massage roller assemblies, see, e.g., U.S. Patent Publication No. 2021/0128399 (the “'399 publication”), and other devices, such as those taught in U.S. Patent Publication No. 2021/0401663 and U.S. Patent Application No. 63/240,042, filed Sep. 2, 2021 and other therapeutic, wellness or exercise related devices, where a plurality of technologies come together to provide therapy to a user. See also U.S. Patent Publication No. 2020/0289365. The entirety of all of the applications listed above are incorporated by reference herein. For example, the technologies that can be included in the inventive smart mat assembly can include vibration, LED light, photobiomodulation, percussion or percussive therapy, microcurrent, infrared, heat, pulsed electro-magnetic field (PEMF), sound therapy, etc.

Furthermore, the smart mat assembly can include wireless connectivity and can connect to a digital platform (and one or more associated apps) where any or all of the above discussed technologies, devices and/or products (e.g., Theragun and/or Therabody products) can interact, be controlled or used. The smart mat assembly can be utilized as a central device or product that brings all the products together and is the core of the Therabody wellness experience.

In a preferred embodiment, the smart mat assembly includes the ability to learn from or collect data about the user's body, movements, etc. while the user is exercising, stretching, meditating or the like to be able to provide a personalized recovery treatment combining embedded technologies, such as one or more of vibration, heat, cold, sound, smell and personalized protocols or routines that can be communicated to other devices, such as a percussive therapy device for a complete recovery session.

It will be appreciated that the smart mat enhances the fitness and wellness routine while the user continues doing what they currently do (e.g., yoga, exercise, etc.), thus allowing the gathering of data and information via sensors and other components to aid the user in the recovery process. The smart mat assembly can be the size of a standard yoga mat or it can be larger or smaller in either width, length or thickness.

The smart mat assembly can be the center of the Therabody wellness routines, a place for a user to reset their body and prepare it for whatever comes next (e.g., sleep, concentration, relaxation, performance, etc.). In a preferred embodiment, the smart mat assembly includes gamma frequency vibration technology, which includes an array of smart interconnected motors that adapt to a user's body and provide tension relief or other therapeutic or desired benefit as the user lays down and relaxes or otherwise utilizes the smart mat assembly.

The smart mat assembly can be used for regular fitness (e.g., exercises), yoga, and software application or “app” guided wellness protocols, for example to reset one's body. See the '307 publication. The smart mat assembly can also be used for interaction with or connection to other virtual class platforms (fitness apps) to provide live feedback through vibration (e.g., Apple fitness, Mirror, Strava, Peloton and Headspace). In such a case, the smart assembly may be used as the interactive post-workout platform for the above-mentioned fitness apps.

In a preferred embodiment, the present invention provides or includes full body vibration that provides deep muscle relaxation and tension relief, specific frequencies that are utilized to resonate with the user's body or brain to generate specific mind states. Such vibration frequencies can be used to provide motor vibration patterns and induce those mind states from concentration to sleep. The app can provide specific protocols and or routines that are designed to provide benefits to the user, such as recovery, relaxation, therapy, etc. The inclusion of a large number of motors dispersed throughout the smart mat assembly allows the mat to provide a large variety of treatments and patterns. For example, a flow mode can include turning motors on and off from the bottom of the mat at the user's feet to the top of the mat near the user's head and can utilize specific frequencies of vibration.

A pressure mode can include sensors or the like that are embedded in or otherwise distributed throughout the mat such that the pressure points are detected. For example, the highest pressure point or area can be detected and the motors in that area can be turned on to provide an intense focused treatment.

A balance mode can include detection (via sensors or the like) of whether the user's weight is evenly or unevenly distributed. If the weight is considered unevenly distributed beyond a predetermined amount, the motors in a certain location vibrate to help guide the user to the balanced position where the weight is evenly distributed within a predetermined amount.

The smart mat assembly may also include vibration mesh to guide movements and provide muscle relaxation/recovery, heating and cooling elements distributed throughout or on the top surface of the mat, pressure meters or sensors that allow the mat to help control or guide a user's posture and/or balance, speakers to guide meditation and stretching sessions and protocols, heart rate sensors, temperature sensors (such that a user's temperature can be determined, e.g., through the feet), infrared technology for enhanced recovery, Bluetooth or other wireless connectivity to interact with one or more apps and any of the other devices discussed herein. The mat can include the necessary PCB(s), controller and wiring to control and communicate with (data communication and/or electrical communication) the motors, switches, sensors, heating and cooling elements, speakers, etc. and to connect to the app or other devices.

In a preferred embodiment, the smart mat assembly includes connectivity with other apps like Apple Fitness+, Strava, Mirror and Peloton that can provide or help collect additional information, which can be collected and stored in a database either remotely or locally. For example, the Apple fitness value add is the connectivity between the platform and the smartwatch. The smart mat assembly may be one additional element that provides live input to the Apple fitness platform.

The present invention can also include interactive Therabody media content. Wellness content can be created that is enhanced by the data the smart mat assembly gathers in real-time to create a wellness experience. Taking protocols to the next level.

In a preferred embodiment, the mat assembly also includes sensors. The sensors can be located anywhere within the mat, between layers, embedded in layers, positioned on top of a layer, etc. The sensors can be part of an “electronics layer” that includes the sensors and other electronics related components (wires, etc.) needed to connect components, such as the vibration motors, switches, sensors, controller/control module, etc. Preferably, the sensors are embedded in or positioned on the mat so that the sensor or sensors are positioned adjacent to or in contact with the user, when the user is on the mat. Any type of sensor or any of the sensors discussed herein can be included. The mat (or any of the other layers) may also include cabling or wiring (and defined pathways therefor) for electrical or data connection or communication between the various components, as necessary.

In a preferred embodiment, the mat assembly includes wireless communication or connectivity (e.g., BLUETOOTH®) so that it can communicate with a software application on a mobile device, such as a phone to provide a “smart” mat system. For example, see a similar smart system or intelligence method disclosed in U.S. Publication No. 2021/0022955 (the “'955 publication”), the entirety of which is incorporated by reference herein. The wireless communication device can be housed on a PCB that may or may not be a part of a control module/controller and that is also in electrical and/or data communication with the vibration devices and various sensors.

The mat assembly may include vibration motors, modules, assemblies or devices that are disposed anywhere throughout the mat assembly and can be positioned to cover or affect different body parts or muscles.

The control assembly may include a plurality of buttons or switches thereon for controlling the mat and the various modes. For example, the control assembly may include a button that turns the device on and off, button(s) for controlling the time or duration, button(s) for changing modes, and button(s) for controlling the vibration devices and turning them on and/or off for various body parts and LED lights related thereto (such as charging indicator(s) and time light indicator(s)). Some of the features are controlled by multiple pushes of the associated button.

In a preferred embodiment, the mat assembly includes wireless communication so that it can communicate with a software application on a mobile device, such as a phone to provide a “smart” mat assembly or system. The wireless communication device can be included on a PCB in the control module that is also in electrical and/or data communication with the vibration devices and various sensors.

The inventive mat assembly and the associated software, controller, remote software application and other components or features discussed herein may be embodied in a therapy system that includes the mat assembly and includes a network interface, an intelligence engine configured to receive manual capture data and real-time tracking data from the mat assembly, receive remote data from at least one remote data source, and generate recommendation data comprising a recommended protocol to be performed by the mat system. The recommendation data is generated from at least one of demographic data, activity data comprising prior use of the mat assembly or other related devices, temporal data comprising timing of use of the mat assembly, analytics data corresponding to use of the mat assembly, and biometric data, received from the manual capture data, the real-time tracking data, and the remote data. The recommendation data comprises at least one of a behavior modification recommendation and a wellness insight. The recommendation data is generated based at least in part on first aggregated data from at least one of the demographic data, the activity data, the temporal data, the analytics data, and the biometric data of a user stored on a remote server. The recommendation data is generated based on second aggregated data from the remote data comprising datasets from users within segmented populations to discover associations between behaviors and behavioral outcomes to optimize the behavior modification recommendation. The recommendation data is generated from a weighted scoring determination comprising at least one of a recovery determination score, a wellness determination score, and a behavior determination score. The recommended protocol includes at least two mat assembly routines, each comprising a series of steps configured to be performed in a predetermined order, and, based on the weighted scoring determination, the first aggregated data, and the second aggregated data, one or more of the at least two mat assembly routines is prioritized, and one or more of the series of steps of the at least two mat assembly routines is modified to improve the recovery determination score, the wellness determination score, and the behavior determination score.

The recommendation data may be provided to the mat assembly. The recommendation data may include a user notification of at least one of the recommended protocol, a change to the recommended protocol, and a behavior recommendation. The demographic data may include at least one of a biological parameter of a user, an age of the user, a height of the user, and a weight of the user. The activity data may include at least one of a type of exercise activity, a volume of the exercise activity, an intensity of the exercise activity, and a trend in activity parameters over time. The temporal data may include at least one of an absolute time of an exercise routine of a user and a relative time of the exercise routine to a predetermined event. The analytics data may include at least one of a duration of use, an intensity of use, a sets of vibration motors of use. The biometric data may include at least one of a heart rate of a user, a heart rate variation of the user, sleep metrics of the user, a temperature of the user, and imaging data comprising at least one of a thermographic image of a user, an ultrasound of the user, and an x-ray image of the user.

The weighted scoring determination may include a poor recovery score indicating that increased recovery is needed for a specified body part of the user, and a first routine of the one or more of the at least two mat assembly routines is prioritized to treat the specified body part of the user or provide a specified recommended therapy (of any of the therapies discussed herein) in accordance with the poor recovery score. The weighted scoring determination may include a poor recovery score indicating that increased recovery is needed for the user or a specified body part of the user, and the one or more of the series of steps of the at least two mat assembly routines is modified to treat the specified body part of the user or provide a specified recommended therapy in accordance with the poor recovery score. The weighted scoring determination may include a recommendation based on data obtained from the second aggregated data to treat the specified body part of the user, and data obtained from the first aggregated data indicating that increased recovery is needed for the specified body part of the user or provide a specified recommended therapy.

The invention, together with additional features and advantages thereof, may be best understood by reference to the following description.

### BRIEF DESCRIPTION OF THE DRAWINGS

The invention may be more readily understood by referring to the accompanying drawings in which:

FIG. 1 is a perspective view of a mat assembly in accordance with a preferred embodiment of the present invention;

FIG. 2 is a cross-section take along line 2-2 of FIG. 1;

FIG. 3 is a cross-section of the mat assembly including switches;

FIG. 4 is a schematic plan view of a mat assembly;

FIG. 5 is a cross-section of the mat assembly in the area of the removable battery;

FIG. 6 is a perspective schematic view showing the mat assembly in the standard position or mode;

FIG. 7 is a perspective schematic view showing the mat assembly in the folded position or mode;

FIG. 8 is a perspective schematic view showing the mat assembly in the chair position or mode;

FIG. 9 is a perspective view showing the battery exploded from the mat;

FIG. 10 is a schematic view of the graphical user interface with the mat depicted thereon;

FIG. 11 is a system block diagram showing structure of a vibration therapy system; and

FIG. 12 is a flow diagram depicting a method of providing therapeutic effect using a mat assembly utilizing an intelligence engine in accordance with an embodiment of the present invention.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the-disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

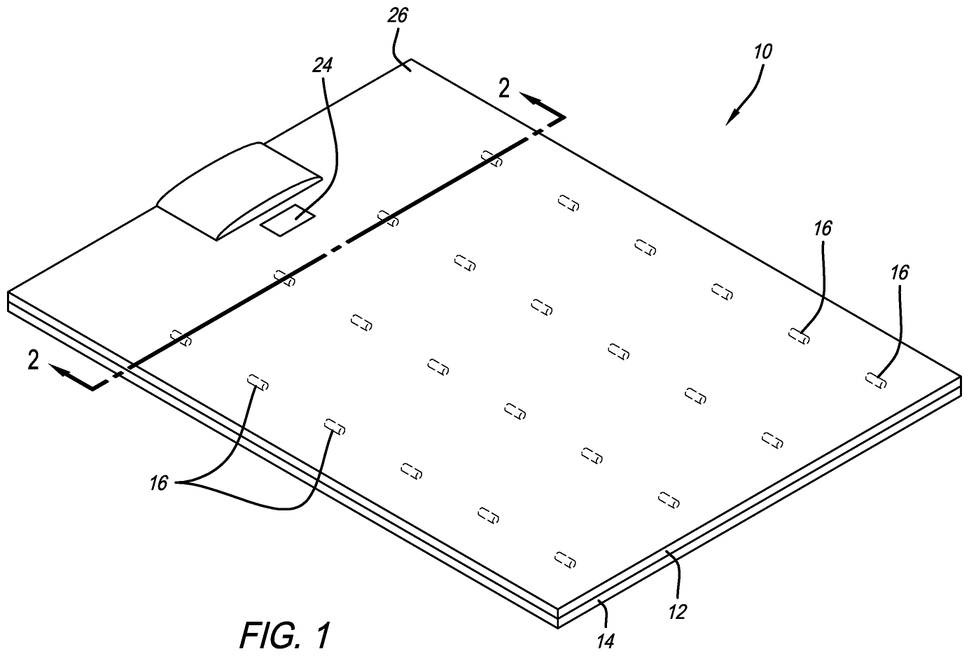

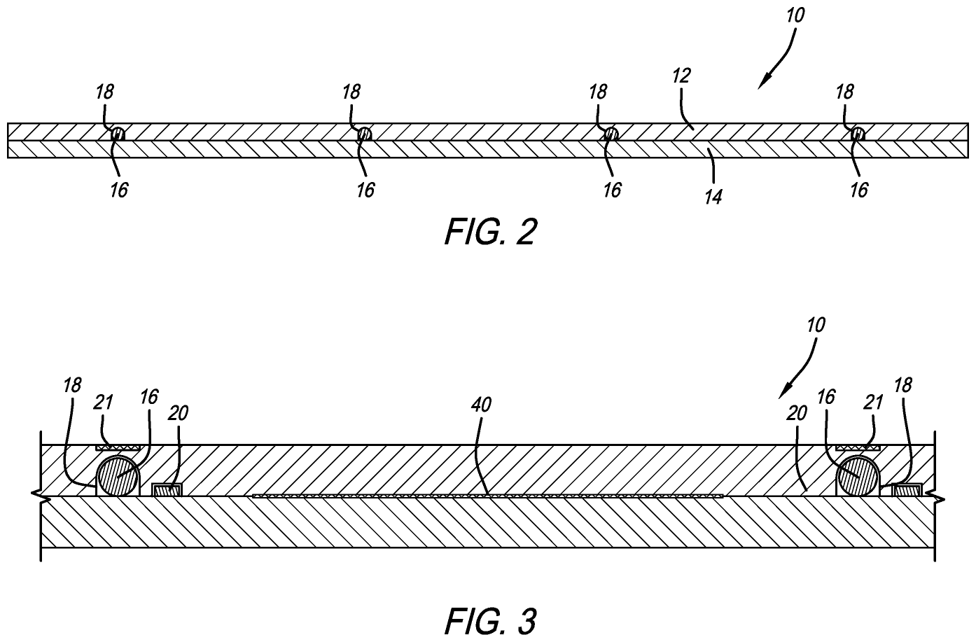

FIGS. 1-10 show embodiments of a mat assembly 10 in accordance with a preferred embodiment of the present invention. As shown in FIG. 1, in a preferred embodiment, the mat assembly 10 generally includes a top layer 12, a bottom layer 14 and a plurality of vibration members or motors 16. The vibration motors 16 can be sandwiched between the top and bottom layers 12 and 14 or can be embedded in one or the other of the top and bottom layer. In a preferred embodiment, the vibration motors 16 are cylindrical motors, however, the vibration motors can be any type of vibration member or motor.

In a preferred embodiment, the top and bottom layers are made of a material such as polyurethane rubber and the vibration motors 16 are positioned in recesses 18 defined in the top or bottom layer or sandwiched therebetween. As shown in FIG. 3, a plurality of switches 20 can be included to switch the motors on and off as desired. The mat assembly 10 can include a switch for each vibration motor or a single switch can control a plurality of vibration motors. Preferably, the vibration motors 16 are positioned close to the top surface (the use surface) of the top layer 12 so they provide as much vibration upwardly (as opposed to downwardly) as possible, but without making the user feel uncomfortable when stepping on the motors, switches or other related electronics with their bare feet.

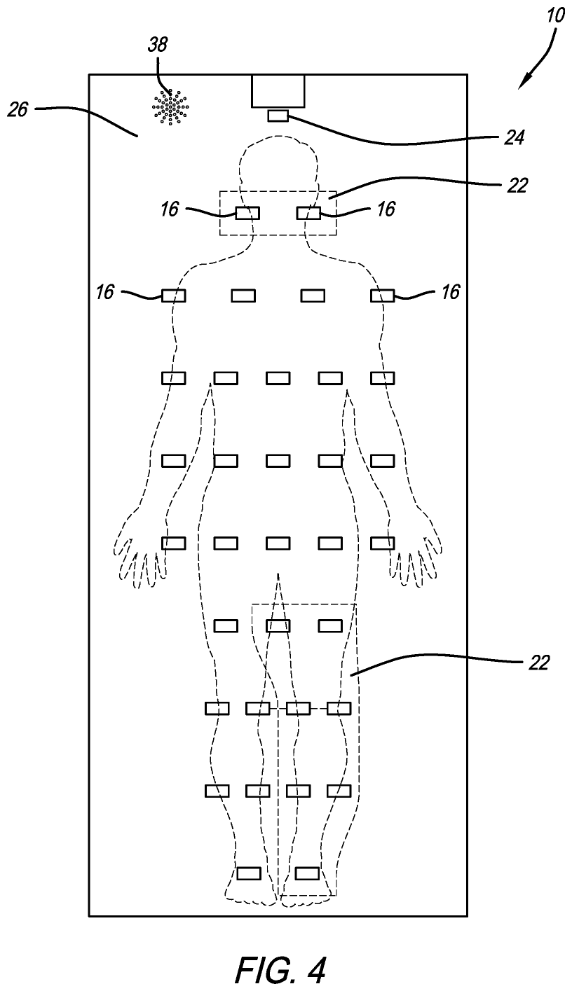

In a preferred embodiment, the switches 20 can be piezoelectric switches. The switches 20 allow selective vibration motors 16 to be turned on and off, which allows the smart mat assembly to follow or detect the user's movements and turn on the vibration motors that are in direct contact with or positioned under the user's body. Sensors 21 can be included that detect pressure, weight, etc. The sensors 21 can then communicate to the switches to turn the motors on (if a body part is positioned over the vibration motor or placing pressure on the area of the mat associated with that particular vibration motor) or off (if a body part is not positioned over the vibration motor or not placing pressure on the area of the mat associated with that particular vibration motor or set of vibration motors). See FIG. 4, which shows a user laying on a mat assembly 10 and showing the vibration motors 16 that are positioned under the user and therefore may be turned on. FIG. 4 also shows rows or sets 22 of motors that align with different areas of a user's body. The sets 22 can be switched on and off as desired or as controlled in different protocols or the like. The sets 22 can comprise any number of different vibration motors at any position on the mat. A set can include a single vibration motor or any number of vibration motors.

FIG. 1 shows six rows or vibration motors 16 that align with different areas of the body (or are in different sections or portions of the mat), and FIG. 4 shows nine rows or sets vibration motors 16. Each row can be considered a set 22. In another embodiment, a set can include vibration motors 16 in more than one row. For example, see sets 22 in FIG. 4, one that is associated with the user's left leg and another that is associated with the neck. The sets 22 can be turned on or off as desired to provide vibration to different body parts. For example, in FIG. 4, row one provides vibration to the head and/or neck, row two provides vibration to the shoulders, row three provides vibration to the arms and upper back, row four provides vibration to the lower back and hands, row five provides vibration to the waist, row six provides vibration to the upper legs, row seven provides vibration to the knee areas, row eight provides vibration to the lower legs and row nine provides vibration to the feet.

In a preferred embodiment, the present invention may provide or include full body vibration that provides deep muscle relaxation and tension relief, specific frequencies that are utilized to resonate with the user's body or brain to generate specific mind states. Such vibration frequencies can be used to provide motor vibration patterns and induce those mind states from concentration to sleep. Frequency bands that can be used and the brain states they can help create. For example, Gamma frequency band vibration, which is above 35 Hz can help provide a concentration state; Beta frequency band vibration, which is between 12-35 Hz can help provide anxiety dominant, active, external attention and relaxed state(s), Alpha frequency band vibration, which is between 8-12 Hz can help provide very relaxed, passive attention state(s); Theta frequency band vibration, which is between 4-8 Hz can help provide deeply relaxed, inward focus state(s); and Delta frequency band vibration, which is between 0.5-4 Hz can help provide a sleep state.

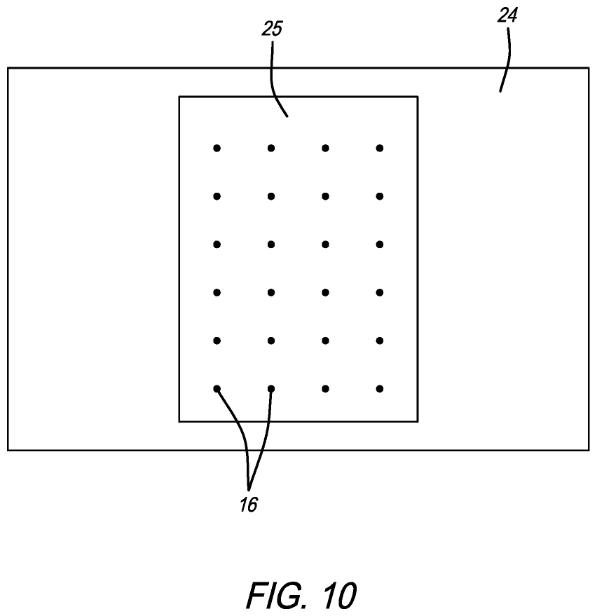

The embodiment shown in FIGS. 1-3 and 5-10 show an embodiment of the present invention that is preferably smaller than a standard yoga mat, whereas the embodiment shown in FIG. 4 is generally the same size or larger than a standard yoga mat. In a preferred embodiment, the mat assembly 10 can be Bluetooth enabled (include wireless connectivity), include USB-C charging (or other type of battery charging), and include different vibration modes (any of those discussed herein). For example, the smart assembly 10 may include a first mode that includes all vibration motors 16 running at low speed, a second mode that includes all vibration motors 16 running on high speed and a third mode that includes the vibration motors 16 running on a vibration wave. In a preferred embodiment, as shown in FIGS. 1 and 10, the user interface 24 is located on the main body portion 26 of the mat or pad and can include control buttons, a screen (may or may not be a touch screen), and an on/off switch or the like. In a preferred embodiment, the mat assembly 10 is wirelessly connectable to a mobile device, such as a phone or tablet.

As discussed herein, the mat assembly 10 can also include any or all of the following types of therapies, LED light, photobiomodulation, percussion or percussive therapy, microcurrent, infrared, heat, pulsed electro-magnetic field (PEMF), sound therapy, etc. For example, see the speaker 38 in FIG. 4 and the heating/cooling element 40 in FIG. 3.

In the embodiment shown in FIG. 1, the main body portion 26 of the mat (sometimes referred to herein simply as the “mat”) is shorter than a typical yoga mat. For example when a user lays on the mat, the mat may only extend from the user's head to their hip area. In an exemplary embodiment, the main body portion 26 is 30 inches long, 22 inches in width and 10 mm thick. In an exemplary embodiment, the mat and/or the layers of the mat can be made of PU leather. It will be appreciated that all measurements in the drawings or description are exemplary and are not limiting.

In a preferred embodiment, as shown in FIG. 10, the graphical user interface 24 may show a depiction of the mat (referred to herein as the “graphical mat” and numbered 25) and allows the user to move their finger on the graphical mat and the vibration motors within the mat will turn on and off in accordance with the user's finger movement on the graphical mat. In other words, the user is guiding the massage, such that the vibration motors 16 turn on or are actuated where desired. The vibration motors can also be grouped into sets or sections on or of the graphical mat such that when a section of the mat is touched, the actual vibration motors within that section are actuated.

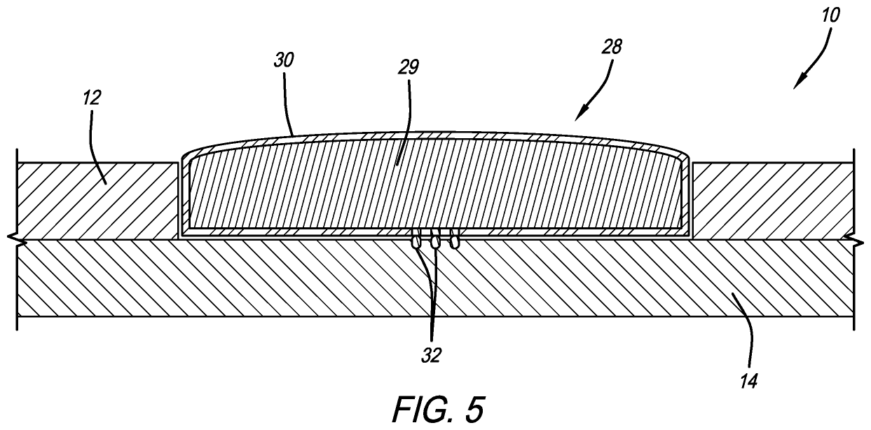

FIG. 5 shows the removable battery pack 28 that, in a preferred embodiment, includes the battery 29 covered or encased in rubber or a rubber layer 30 so that the battery pack 28 is comfortable and/or cushioned for a user to lay on. FIG. 5 also shows the electrical contacts 32 where the battery pack 28 is electrically connected within the mat assembly 10.

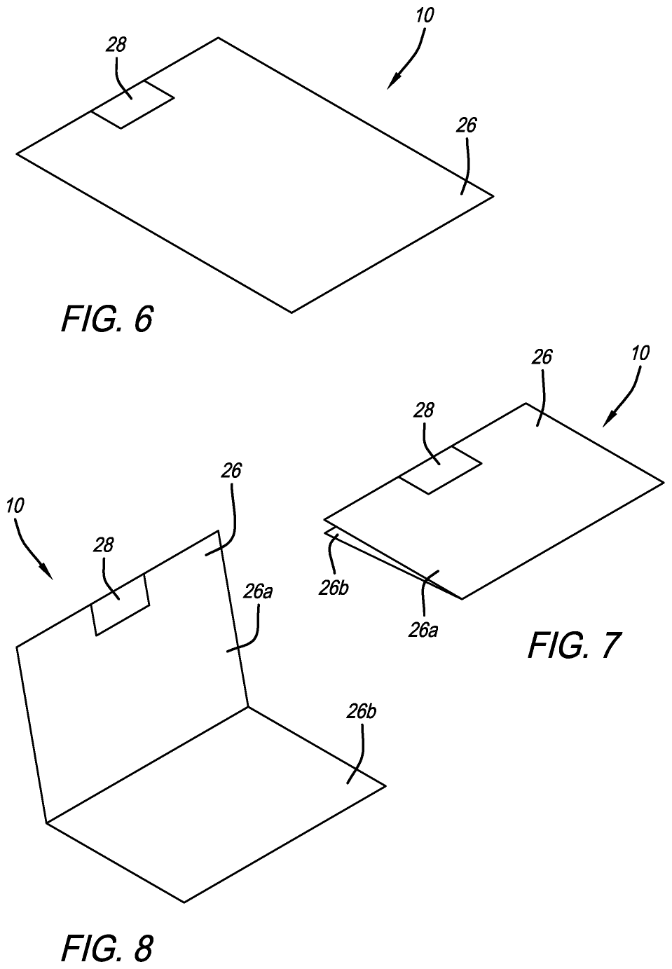

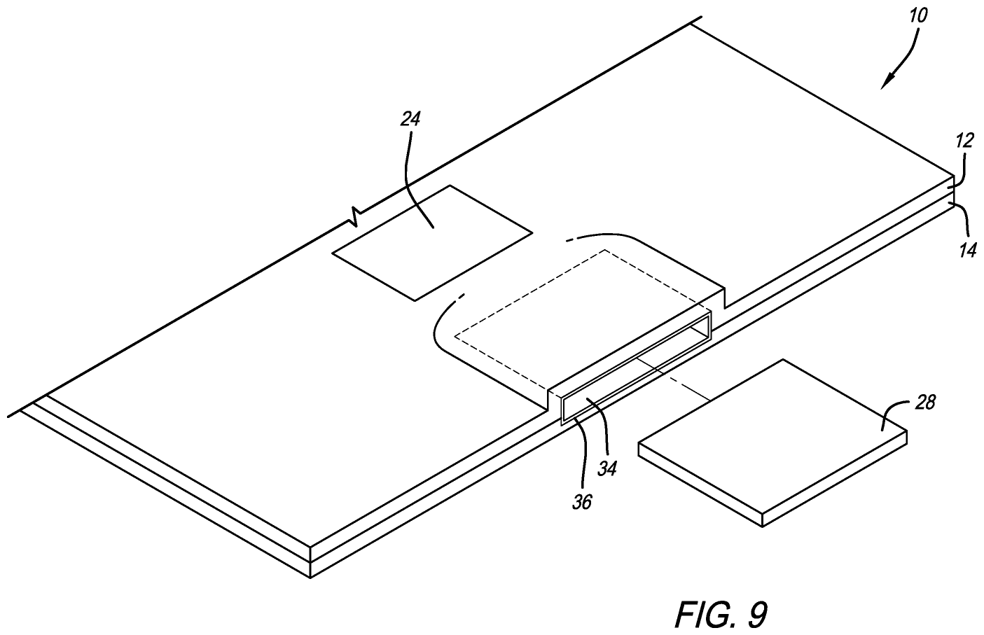

FIGS. 6-8 show different use cases and possible orientations or modes of use of the mat assembly 10. FIG. 6 shows the mat assembly 10 oriented in the standard position or mode where the entire mat is laid flat against the ground and can be utilized where the user is laying down, e.g., for post-workout treatment, for therapeutic purposes and for mediation. FIG. 7 shows the mat assembly 10 oriented in the folded position or mode that can be used while the user is standing, e.g., while using a standing desk and for other feet treatment. FIG. 8 shows mat assembly 10 in the chair position or mode that can be used where the user is seated, e.g., in a desk chair, car seat or airplane seat.

FIG. 9 shows a preferred embodiment of a removable battery pack 28 that can be inserted into a battery cavity 34 that is positioned between the top and bottom layers 12 and 14 of the mat. The battery pack 28 is inserted into a battery receiver 36 that includes the battery cavity 34 and is electrically connected to the electrical system of the mat. Preferably, the battery pack includes a spring-based or other release system.

In a preferred embodiment, the mat assembly 10 includes the sensors 21 discussed above that sense where a user is placing weight (e.g., stepping on the mat or laying on the mat) and the vibration motors in the areas where the user is placing weight are switched on. This feature can save battery life by not operating vibration motors in areas where the user is not located and can also reduce noise.

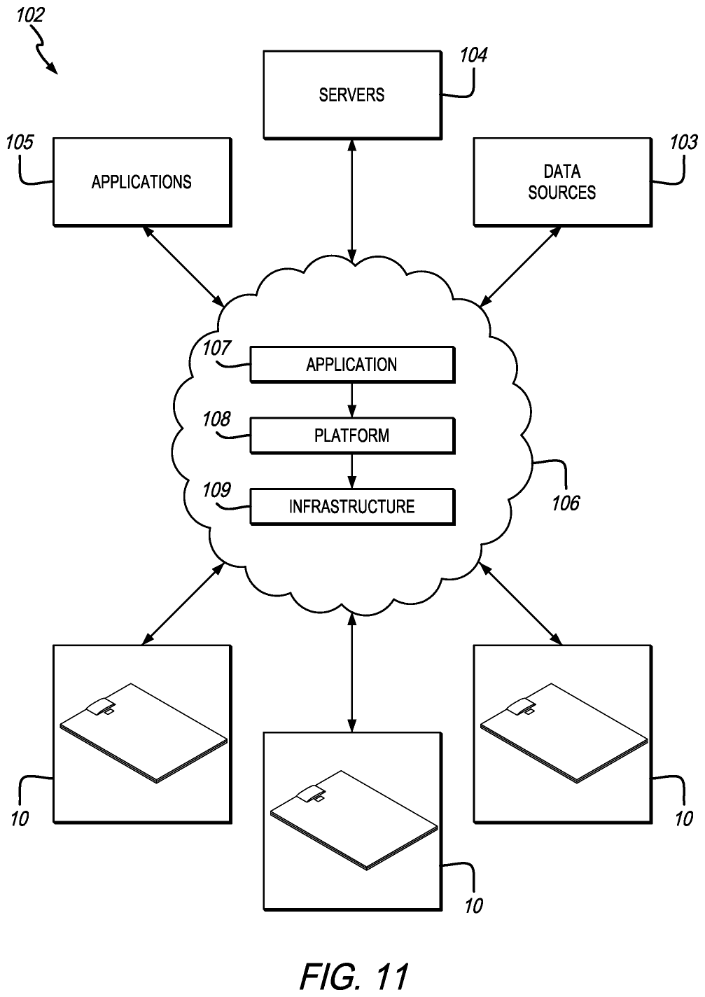

FIG. 11 is a system block diagram showing structure of a mat assembly system 102. The mat assembly system 102 includes one or more mat assemblies 10, data sources 103, servers 104, applications 105, and a cloud 106.

The data sources 103 include, for example, online or cloud-based data sources of health and wellness information. The health and wellness information may be aggregated data from a number of unorganized sources upon which statistical analysis can be done. The data sources 103 may also include biometric information from wearable biometric devices, such as, for example, Biostrap wearable devices, Apple® wearable devices, Whoop® wearable devices and the like. The data sources 103 may include information from Apple's Apple Health application, MyFitnessPal application, and the like.

The servers 104 and applications 105 are well known to one of ordinary skill in the art. The servers 104 may include structure configured to facilitate processing and data storage and transfer. The applications 105 may be standalone applications configured to be executed on a smart device, a standalone computer, a laptop, an entertainment center, or other computing devices.

In this embodiment, the cloud 106 includes an application 107, a platform 108, and an infrastructure 109. For example, the application 107 may include a variety of applications configured to execute all or portions of the functions of an intelligence engine in connection with the platform 108 and the infrastructure 109. One of ordinary skill in the art would understand that the cloud 106 and its constituents therein is only one way to depict a cloud-based computing system and there are a variety of other ways to depict the same without departing from the scope of the present invention.

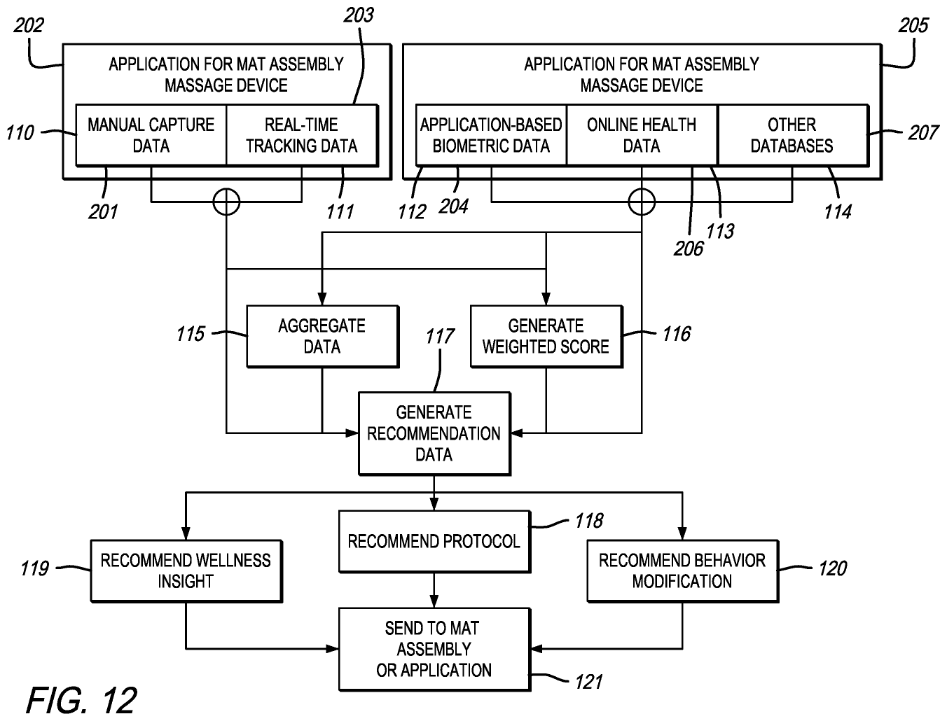

FIG. 12 is a flow diagram depicting a method of providing therapeutic effect using a mat assembly 10 utilizing the intelligence engine in accordance with an embodiment of the present invention.

At Step 110, manual capture data 201 is generated. The manual capture data 201 is, for example, data input via the touch screen of a mobile device or tablet or the user interface of the mat. An application 202 running on a smart device associated with the touch screen may prompt a user to input answers to questions regarding health, wellness, or other parameters useful to provide recommendation data to the user. Alternatively, though not shown in connection with FIG. 12, a user may input data directly into the mat assembly 10, which may then be transferred wirelessly to be used by the intelligence engine. Also, the application 202 can be associated with other products within the same brand, such as Therabody (e.g., see the '955 publication). See also the garment assembly and associated data disclosed in U.S. patent application Ser. No. 17/678,924, the entirety of which is incorporated by reference herein in its entirety. Data from all of the different devices or products can be aggregated to provide the user with various recommendations, suggestions, protocols, routines, etc.

At Step 111, real-time tracking data 203 is generated. In the embodiment depicted in FIG. 12, an application 202 configured to wirelessly connect to the mat assembly 10 monitors and stores real-time tracking data of a user's use of the mat assembly 10. In an embodiment, the application 202 transmits the real-time tracking data to a cloud-based computing system such as that shown in FIG. 11. In other embodiments, a standalone computing system may be utilized.

At Step 112, application-based biometric data 204 is provided via one of a remote data sources 205. At Step 113, online health data 206 is provided via another one of the remote data sources 205. At Step 114, data from other databases 207 is provided via another one of the remote data sources 205. One of ordinary skill in the art would understand that the remote data sources 205 may include the data sources 103.

One of ordinary skill in the art would understand that the various input data described herein may be substituted for the particular input data described in connection with FIG. 12 without departing from the scope or spirit of the present invention.

At Step 115, all or portions of the manual capture data 201, real-time tracking data 203, application-based biometric data 204, online health data 206, and data from the other databases 207 are aggregated. One of ordinary skill in the art would understand the methodology for data aggregation, and as more fully described herein. In an embodiment, third party data and user data are aggregated separately. In another embodiment, all data is aggregated.

At Step 116, a weighted score is generated based on all or portions of the manual capture data 201, real-time tracking data 203, application-based biometric data 204, online health data 206, and data from the other databases 207. The weighted score may include a recovery determination score, a wellness determination score, and a behavior determination score. As an example, the recovery determination score includes a determination of how long a user's HB returned to a restorative state. Depending on the application's parameters, the score could, for example, determine that a Recovery Score is Poor, as described more fully below in Table 1. As another example, a wellness determination score includes a determination of dietary intake and trends to determine an overall wellness score. Depending on the application's parameters, the score could, for example, determine that a data input regarding dietary intake was within predetermined parameters, thereby increasing the user's wellness determination score. As another example, a behavior determination score includes a determination of sleep metrics and trends to determine an overall behavior determination score. Depending on the application's parameters, the score could, for example, determine that a Sleep Metrics score was Poor, as described more fully below in Table 1.

At Step 117, recommendation data is generated based on all or portions of (1) the aggregated data (2) the weighted score and (3) all or portions of the manual capture data 201, real-time tracking data 203, application-based biometric data 204, online health data 206, and data from the other databases 207. These data may all be combined to generate the recommendation data. Alternatively, only a weighted score is utilized to generate the recommendation data. In yet another alternative, only real-time tracking data 201 is utilized to generate the recommendation data. One of ordinary skill in the art would understand the various data inputs are fluid and may be utilized based on desired parameters for optimum health and wellness.

At Step 118, a recommended protocol is determined as part of the recommendation data. The recommended protocol is, in an embodiment, obtained from a library of protocols. For example, see FIGS. 26-29 in the '955 publication, which show various protocols that may be obtained from the library of protocols for a percussive massage device. Similar protocols, including time, speed, motor or motor set, pattern (e.g., continuously on, wave, pulse, etc.) and other features can be included in the protocols or routines for the mat assembly. In another embodiment, the recommended protocol is synthesized from available data, i.e., a “bespoke” routine synthesis suitable for a particular user. Table 1 below depicts how routines of the protocol may be prioritized and/or steps within each of the routines may be modified to accommodate various data inputs. For example, the recommended protocol may consist of more than one routine.

At Step 119, a wellness insight is recommended as part of the recommendation data. The wellness insight, for example, may be based on the weighted score that determines that the user's dietary intake is poor and thus, would provide an insight that may assist the user to modify their dietary intake. Other examples are within the scope of the present invention.

At Step 120, a behavior modification is recommended as part of the recommendation data. The behavior modification, for example, may be based on the weighted score that determines that a user's Sleep Metrics are Poor, thereby prompting a behavior modification notification to the user to alert the user about his or her poor sleep habits.

At Step 121, one or more of the recommended protocol, wellness insight, or behavior modification is provided to the mat assembly 10 or the application 202. Preferably, the user of the mat assembly 10 is notified in accordance with the recommendation data.

Table 1 below provides an example of input data and output data for a particular scenario in accordance with a preferred embodiment.

INTELLIGENCE ENGINE

| INPUT DATA | OUTPUT DATA |
|---|---|
| Female | Modification of steps in routines |
| 57 | Modification of steps in routines |
| Activity = Yoga | Prioritization of specific routines and |
| | personalized notifications |
| Duration = 51 minutes | Prioritization of specific routines and the |
| | modification of steps within them |
| Trends = X % Longer than | Prioritization of specific routines and the |
| normal | modification of steps within them |
| Time = Evening | Prioritization of second series of routines |
| | and personalized notifications |
| Time = Within 2 hours of | Prioritization of specific routines, and |
| activity completion | personalized notifications |
| Recent Vibration Therapy = | Modification of steps in routines and |
| Short + Infrequent | highlighting of insights |
| Recovery Score = Poor | Prioritization of specific routines, the |
| | modification of steps within them, |
| | personalized notifications, and highlighting |
| | of insights |
| Sleep Metrics = Poor | Prioritization of specific routines, the |
| | modification of steps within them, |
| | personalized notifications, and highlighting |
| | of insights |

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶6, other aspects may likewise be embodied as a means-plus-function claim, or in other forms, such as being embodied in a computer-readable medium. (Any claims intended to be treated under 35 U.S.C. § 112, ¶6 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A mat assembly comprising:
a mat that includes a top layer and a bottom layer,
a plurality of vibration motors positioned between the top layer and the bottom layer,
a plurality of sensors associated with the mat,
a removable battery pack, and
a controller,
wherein a first set of vibration motors within the plurality of vibration motors is configured to be actuated when a first one of the plurality of sensors is actuated.

2. The mat assembly of claim 1 wherein the removable battery pack is received in a battery receiver that is positioned between the top and bottom layers.

3. The mat assembly of claim 1 wherein the removable battery pack includes an outer layer made of a rubber material and is positioned in a battery opening defined in the top layer.

4. The mat assembly of claim 1 further comprising a wireless connection device.

5. The mat assembly of claim 4 further comprising a software application that is configured to control the operation of the smart mat.

6. The mat assembly of claim 1 further comprising a graphical user interface positioned on the top layer.

7. The mat assembly of claim 6 wherein the graphical user interface includes a touch screen that is configured to display a graphical mat, wherein the plurality of vibration motors are controllable by touching the graphical mat.

8. The mat assembly of claim 1 further comprising sound therapy.

9. The mat assembly of claim 1 further comprising heat or cold therapy.
